(19) United States
(12) Patent Application Publication
(10) Pub. No.: US 2023/0298712 A1
(43) Pub. Date: Sep. 21, 2023

(54) SERVER AND METHOD FOR PROVIDING USER-SOVEREIGN MEDICAL DATA SHARING AND TRANSACTION SERVICE

(71) Applicant: ELECTRONICS AND TELECOMMUNICATIONS RESEARCH INSTITUTE, Daejeon (KR)

(72) Inventors: Bumhwi KIM, Daejeon (KR); Kyu Hyung KIM, Daejeon (KR)

(21) Appl. No.: 17/945,227

(22) Filed: Sep. 15, 2022

(30) Foreign Application Priority Data
Mar. 16, 2022 (KR) 10-2022-0032605

(51) Int. Cl. G16H 10/60; G06Q 30/02; G16H 40/63; G06F 21/62; G06F 21/60; G06F 21/10

(52) U.S. Cl. CPC G16H 10/60; G06Q 30/0236; G16H 40/63; G06F 21/6245; G06F 21/602; G06F 21/10; G06F 2221/0713

(57) ABSTRACT

There is provided a server for providing a user-sovereign medical data sharing and transaction service. The server includes a medical data collection unit configured to collect medical data generated from at least one of a user and a medical institution, a blockchain unit configured to store the collected medical data in a blockchain network while operating in conjunction with the blockchain network, and an artificial intelligence unit configured to read, from the blockchain network, medical data corresponding to the use request when receiving a use request for the stored medical data from another user, de-identify the read medical data, and provide the de-identified medical data to the other user.

### CROSS-REFERENCE TO RELATED APPLICATION

This application claims priority to and the benefit of Korean Patent Application No. 10-2022-0032605, filed on Mar. 16, 2022, the disclosure of which is incorporated herein by reference in its entirety.

### BACKGROUND

### 1. Technical Field

The present disclosure relates to a server and method for providing a user-sovereign medical data sharing and transaction service.

### 2. Description of Related Art

A patient visits hospitals and health centers for purposes, such as medical treatment, treatment, and examination, and receives health services. Furthermore, when the patient subsequently visits the hospitals and health centers, the patient may smoothly receive desired health services because medical treatment, treatment, and examination records of the patient are kept in the hospitals and health centers.

However, if the patient visits other hospitals and health centers never visited by the patent, the patient has to newly redundantly receive health services, such as medical treatment, treatment, and examination. In order to avoid such redundant services, the patient has to inconveniently request records of health services from hospitals and health centers that the patient has previously visited and to submit a copy of the records to other hospitals and health centers that the patent newly visits. Most of the hospitals and health centers charge a cost for the copy.

Furthermore, practically, even an institution for research purposes very inefficiently obtains medical data by collecting the medical data from a process of newly collecting medical data without using medical data from the previous hospitals and health centers. Medical data, such as health medical examination, is updated in National Health Insurance Corporation every year through de-identification. However, there is a problem in that restricted access is possible in a limited space only when a research plan has to be submitted and expenses have to be paid in order use the medical data.

### SUMMARY

Various embodiments are directed to providing a server and method for providing a user-sovereign medical data sharing and transaction service, which completely change medical data of a user himself or herself into person-centered medical data not medical data based on medical institutions, such as hospitals and health centers, and enable medical data to be limitedly shared and traded, if necessary, not a purpose for storing the medical data.

However, an object to be solved by the present disclosure is not limited to the aforementioned object, and other objects may be present.

A method of sharing and trading user-sovereign medical data according to a first aspect of the present disclosure includes collecting medical data generated from at least one of a user and a medical institution, storing the collected medical data in a blockchain network, receiving a use request for the stored medical data from another user, reading, from the blockchain network, medical data corresponding to the use request, and de-identifying the read medical data and providing the de-identified medical data to the other user.

Furthermore, a server for providing a user-sovereign medical data sharing and transaction service according to a second aspect of the present disclosure includes a medical data collection unit configured to collect medical data generated from at least one of a user and a medical institution, a blockchain unit configured to store the collected medical data in a blockchain network while operating in conjunction with the blockchain network, and an artificial intelligence unit configured to read, from the blockchain network, medical data corresponding to the use request when receiving a use request for the stored medical data from another user, de-identify the read medical data, and provide the de-identified medical data to the other user.

Furthermore, a server for providing a user-sovereign medical data sharing and transaction service according to a third aspect of the present disclosure includes a communication module configured to transmit and receive data to and from a terminal of a user and a terminal of a medical institution, a memory in which a program for providing a medical data sharing and transaction service based on medical data generated from at least one of the user and the medical institution has been stored, and a processor configured to collect and store the medical data when the program stored in the memory is executed and to read medical data corresponding to a use request when receiving the use request for the stored medical data from another user, de-identify the read medical data, and provide the de-identified medical data to the other user.

A computer program according to another aspect of the present disclosure executes the method of providing a user-sovereign medical data sharing and transaction service in association with a computer, that is, hardware, and is stored in a computer-readable recording medium.

Other details of the present disclosure are included in the detailed description and the drawings.

According to one of the aforementioned embodiments of the present disclosure, personalized health services in a medical institution can be provided because medical data can be shared and traded.

Furthermore, there are advantages in that incentives can be provided to a person who provides medical data and entry barriers for artificial intelligence-based research using medical data can be reduced with respect to another user who purchases the medical data.

The effects of the present disclosure are not limited to the above-mentioned effects, and other effects which are not mentioned herein will be clearly understood by those skilled in the art from the following descriptions.

### BRIEF DESCRIPTION OF THE DRAWINGS

FIG. 1 is a diagram for describing a system for providing a medical data sharing and transaction service according to an embodiment of the present disclosure.

FIG. 2 is a functional block diagram of a server for providing a medical data sharing and transaction service according to an embodiment of the present disclosure.

FIG. 3 is a construction diagram of the server for providing a medical data sharing and transaction service according to an embodiment of the present disclosure.

FIG. 4 is a flowchart of a method of sharing and trading user-sovereign medical data according to an embodiment of the present disclosure.

### DETAILED DESCRIPTION

Advantages and characteristics of the present disclosure and a method for achieving the advantages and characteristics will become apparent from the embodiments described in detail later in conjunction with the accompanying drawings. However, the present disclosure is not limited to the disclosed embodiments, but may be implemented in various different forms. The embodiments are merely provided to complete the present disclosure and to fully notify a person having ordinary knowledge in the art to which the present disclosure pertains of the category of the present disclosure. The present disclosure is merely defined by the category of the claims.

Terms used in this specification are used to describe embodiments and are not intended to limit the present disclosure. In this specification, an expression of the singular number includes an expression of the plural number unless clearly defined otherwise in the context. The term “comprises” and/or “comprising” used in this specification does not exclude the presence or addition of one or more other elements in addition to a mentioned element. Throughout the specification, the same reference numerals denote the same elements. The term “and/or” includes each of mentioned elements and all combinations of one or more of mentioned elements. Although the terms “first”, “second”, etc. are used to describe various components, these elements are not limited by these terms. These terms are merely used to distinguish between one element and another element. Accordingly, a first element mentioned hereinafter may be a second element within the technical spirit of the present disclosure.

All terms (including technical and scientific terms) used in this specification, unless defined otherwise, will be used as meanings which may be understood in common by a person having ordinary knowledge in the art to which the present disclosure pertains. Furthermore, terms defined in commonly used dictionaries are not construed as being ideal or excessively formal unless specially defined otherwise.

The present disclosure relates to a server 100 and method for providing a user-sovereign medical data sharing and transaction service.

According to an embodiment of the present disclosure, medical data of a person can be autonomously donated or traded because the medical data is not owned by hospitals and health centers (hereinafter referred to as a “medical institution”), but is managed as user-sovereign medical data.

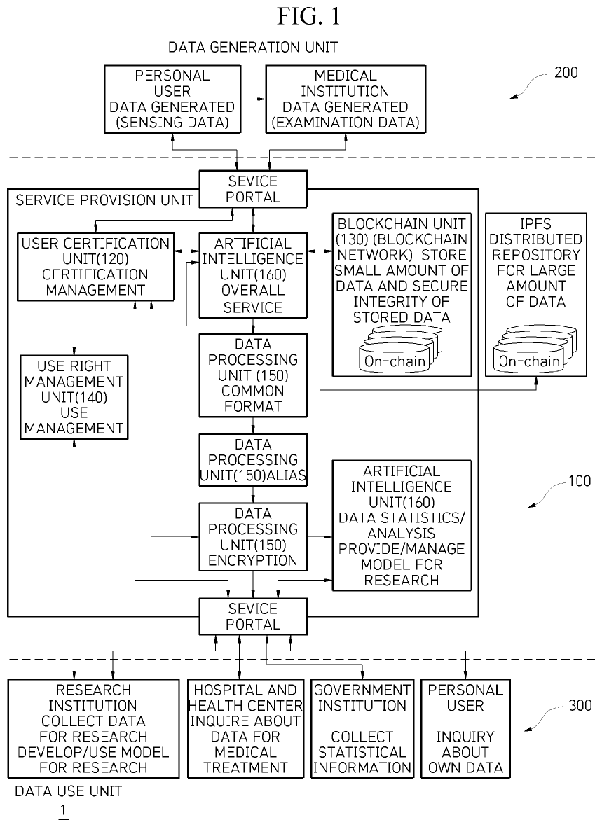

Hereinafter, the server 100 for providing a medical data sharing and transaction service according to an embodiment of the present disclosure is described with reference to FIGS. 1 to 4.

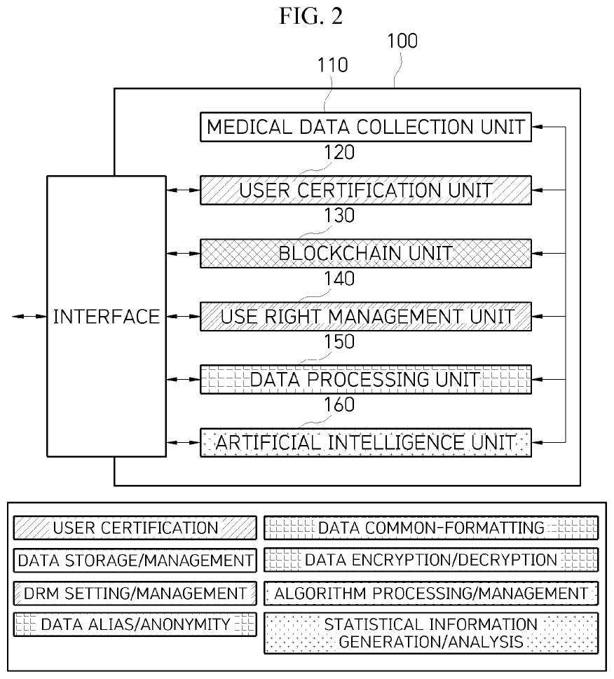

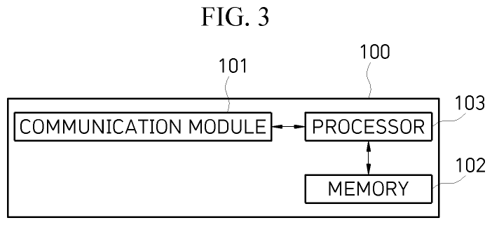

FIG. 1 is a diagram for describing a system 1 for providing a medical data sharing and transaction service according to an embodiment of the present disclosure. FIG. 2 is a functional block diagram of the server 100 for providing a medical data sharing and transaction service according to an embodiment of the present disclosure. FIG. 3 is a construction diagram of the server 100 for providing a medical data sharing and transaction service according to an embodiment of the present disclosure.

Referring to FIG. 1, the system 1 for providing a medical data sharing and transaction service according to an embodiment of the present disclosure basically includes a data generation unit 200, a data use unit 300, and a service provision unit 100. In this case, the service provision unit 100 corresponds to the server 100 for providing a medical data sharing and transaction service (hereinafter referred to as the “server”) in the present disclosure.

The data generation unit 200 is the subject that generates medical data, and corresponds to a personal user or a medical institution such as a hospital or a health center. That is, the data generation unit 200 generates medical data through a user terminal, a medical institution server, or a terminal. In an embodiment, the user terminal may be a smartphone or a wearable device that operates in conjunction with a smartphone. The medical data generated through the user terminal may be sensing data that is collected from a smartphone or a wearable device. Furthermore, the medical data generated through the medical institution server or the terminal may be examination data of a user who visits a corresponding medical institution, or examination data or experiment data that is generated in other research and analysis processes and that is not related to a specific user.

According to an embodiment of the present disclosure, the type of service for dividing users (e.g., a personal user, a user of a hospital, a user of a research institution, and a user of a government institution) with respect to medical data collected by various subjects and providing the users with the service can be differentiated, and access rights of the users can be managed.

An application that is distributed by the server may be previously installed in a terminal of a user. Sensing data or examination data may be provided to the server through the application. When receiving medical data from a user, the server 100 may provide incentives to the user. In this case, the incentives may be a tangible resource or a non-tangible resource, such as incentives that are managed in a blockchain network. The provided incentives may be used to use a service that is provided by the server 100 or may be encashed. In this case, the service means a form of a model developed through the server 100, which has been sufficiently verified and may be provided to the majority within the system 1.

Referring to FIG. 2, the server 100, that is, the service provision unit 100 includes a medical data collection unit 110, a blockchain unit 130, and an artificial intelligence unit 160.

The medical data collection unit 110 collects medical data that is generated from at least one of a user and a medical institution. In this case, the medical data collection unit 110 may collect the medical data with the previous consent of the collection from a user terminal of the user or a medical institution terminal of the medical institution.

In this case, the server 100 may further include a user certification unit 120, and may provide a user certification-related function for the user and the medical institution.

The blockchain unit 130 stores the collected medical data in a given blockchain network while operating in conjunction with the blockchain network. In an embodiment, the blockchain unit 130 may store, in an external distributed repository, medical data that satisfies a preset condition. Furthermore, for integrity verification, the blockchain unit 130 may store, in the blockchain network, only hash information corresponding to the stored medical data. In this case, the preset condition may be at least one of the type and amount of the medical data.

For example, the blockchain unit 130 may store most of collected medical data in an internal blockchain network, and may store a large amount of image-based medical data, such as CT or MRI having a high capacity, in an external distributed repository. Furthermore, for integrity verification, the blockchain unit 130 may store, in the blockchain network, only hash information corresponding to medical data stored in the external distributed repository.

The data use unit 300 may be basically divided into a data utilization unit and a model utilization unit.

In an embodiment, the data utilization unit provides an inquiry function for enabling a personal user to confirm his or her own data that is stored in the server 100. To this end, when receiving an inquiry request for medical data that belongs to medical data stored in a blockchain network and that has been provided by the user, the artificial intelligence unit 160 may read medical data corresponding to the inquiry request from the blockchain network, and may provide the read medical data to the user.

Through such an inquiry function, a user can inquire about medical data at a medical institution that the user has previously visited through a user terminal of the user without an unnecessary behavior, such as visiting a medical institution that the user has previously visited and outputting medical data, and may easily present the medical data to another medical institution. Furthermore, the user can confirm the corresponding medical data in the other medical institution, and can continuously perform medical behaviors, such as medical care and treatment.

In another embodiment, the data utilization unit may provide a function for de-identifying data that has been stored in the server and lending another user (e.g., a research institution or a researcher) the de-identified data for a given period for the purpose of being used for research and development.

To this end, when receiving a use request for medical data from another user for research purposes, the artificial intelligence unit 160 may read medical data corresponding to the use request from a blockchain network, may de-identify the read medical data, and may provide the de-identified medical data to the other user.

In this case, according to an embodiment of the present disclosure, the server 100 may further include a use right management unit 140 and a data processing unit 150 for the de-identification of the medical data. The use right management unit 140 converts the read medical data into digital rights management (DRM) medical data the lending period of which has been designated. The data processing unit 150 converts the DRM medical data into a preset common data format (CDF), de-identifies personal information that is included in the DRM medical data converted in the CDF, and encrypts the DRM medical data in order to prevent hacking into the DRM medical data.

The artificial intelligence unit 160 transmits the DRM medical data that has been encrypted as described above to a demanding institution, that is, the other user that has made the use request.

Next, the model utilization unit provides a function for registering a development model that has been developed through research and development or purchasing and using a previously developed model. That is, the model utilization unit enables a person or a medical field artificial intelligence developer of an institution to pay expenses according to a development purpose and obtain rights to use medical data having a specific range to be obtained.

To this end, the artificial intelligence unit 160 may register a development model that has been developed based on medical data from another user, and may set the registered development model and a previously registered development model so that the registered development model and the previously registered development model can be distributed. In this case, the registered development model may also include a model use method.

Thereafter, when the registered development model from the other user is distributed according to a purchase behavior of another third user, the artificial intelligence unit 160 may provide incentives that are managed in a blockchain network to the other user who has registered the development model. The provided incentives may be used to purchase another model or may be encashed. The purchased model may be modified or supplemented. A version of the completed model may be served and provided. In this case, a level of contribution when the version of the completed model is served is provided to all developers who have participated in the development of the model.

In addition, according to an embodiment of the present disclosure, for the purpose of providing medical data to a government institution, such as Ministry of Health and Welfare, or a research institution, statistical and analysis information reports for the collected medical data may be generated and provided. In this case, a de-identification process other than DRM may be additionally performed on the medical data.

Referring to FIG. 3, the server 100 according to an embodiment of the present disclosure may be constructed to include a communication module 101, a memory 102, and a processor 103.

The communication module 101 transmits and receives data to and from a user terminal and a medical institution terminal. A program for providing a medical data sharing and transaction service based on medical data generated from at least one of a user and a medical institution is stored in the memory 102.

When the program stored in the memory 102 is executed, the processor 103 collects and stores the medical data. When receiving a use request for the stored medical data from another user, the processor 103 reads medical data corresponding to the use request, de-identifies the read medical data, and provides the de-identified medical data to the other user.

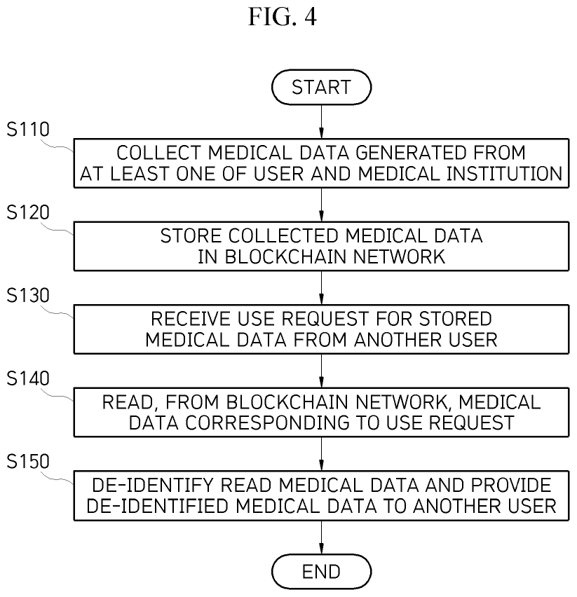

Hereinafter, a method that is performed by the server 100 for providing a medical data sharing and transaction service according to an embodiment of the present disclosure is described with reference to FIG. 4.

FIG. 4 is a flowchart of the method of sharing and trading user-sovereign medical data according to an embodiment of the present disclosure.

First, the server 100 collects medical data generated from at least one of a user and a medical institution (S110), and stores the collected medical data in a blockchain network (S120).

Next, when receiving a use request for the stored medical data from another user (S130), the server 100 reads, from the blockchain network, medical data corresponding to the use request (S140), de-identifies the read medical data, and provides the de-identified to the other user (S150).

In the aforementioned description, steps S110 to S150 may be sub-divided into additional steps or may be assembled into a smaller number of steps depending on an implementation example of the present disclosure. Furthermore, some steps may be omitted, if necessary, and the sequence of steps may be changed. Furthermore, although contents are omitted, the contents of FIGS. 1 to 3 may also be applied to the method in FIG. 4.

The aforementioned embodiment of the present disclosure may be implemented in the form of a program (or application) in order to be executed by being combined with a computer, that is, hardware, and may be stored in a medium.

The aforementioned program may include a code coded in a computer language, such as Golang, C, C++, JAVA, Javascript, Ruby, Python, Solidity, or a machine language which may be readable by a processor (CPU) of a computer through a device interface of the computer in order for the computer to read the program and execute the methods implemented as the program. Such a code may include a functional code related to a function that defines functions necessary to execute the methods, etc., and may include an execution procedure-related control code necessary for the processor of the computer to execute the functions according to a given procedure. Furthermore, such a code may further include a memory reference-related code indicating at which location (address number) of the memory inside or outside the computer additional information or media necessary for the processor of the computer to execute the functions needs to be referred. Furthermore, if the processor of the computer requires communication with any other remote computer or server in order to execute the functions, the code may further include a communication-related code indicating how the processor communicates with the any other remote computer or server by using a communication module of the computer and which information or media needs to be transmitted and received upon communication.

The stored medium means a medium, which semi-permanently stores data and is readable by a device, not a medium storing data for a short moment like a register, cache, or a memory. Specifically, examples of the stored medium include a ROM, a RAM, a CD-ROM, a magnetic tape, a floppy disk, optical data storage, etc., but the present disclosure is not limited thereto. That is, the program may be stored in various recording media in various servers which may be accessed by a computer or various recording media in a computer of a user. Furthermore, the medium may be distributed to computer systems connected over a network, and a code readable by a computer in a distributed way may be stored in the medium.

The description of the present disclosure is illustrative, and a person having ordinary knowledge in the art to which the present disclosure pertains will understand that the present disclosure may be easily modified in other detailed forms without changing the technical spirit or essential characteristic of the present disclosure. Accordingly, it should be construed that the aforementioned embodiments are only illustrative in all aspects, and are not limitative. For example, elements described in the singular form may be carried out in a distributed form. Likewise, elements described in a distributed form may also be carried out in a combined form.

The scope of the present disclosure is defined by the appended claims rather than by the detailed description, and all changes or modifications derived from the meanings and scope of the claims and equivalents thereto should be interpreted as being included in the scope of the present disclosure.

What is claimed is:

1. A method of sharing and trading user-sovereign medical data, the method comprising:
collecting medical data generated from at least one of a user and a medical institution;
storing the collected medical data in a blockchain network;
receiving a use request for the stored medical data from another user;
reading, from the blockchain network, medical data corresponding to the use request; and
de-identifying the read medical data and providing the de-identified medical data to the other user.

2. The method of claim 1, wherein the collecting of the medical data generated from the at least one of the user and the medical institution comprises collecting sensing data of a wearable device of the user or examination data in a medical institution that the user has visited.

3. The method of claim 2, wherein the collecting of the medical data generated from the at least one of the user and the medical institution comprises:
receiving the sensing data or the examination data through an application previously installed in a terminal of the user; and
providing the user with incentives managed in the blockchain network when receiving the medical data.

4. The method of claim 1, wherein the storing of the collected medical data in the blockchain network comprises:
storing, in an external distributed repository, medical data satisfying a preset condition; and
storing, in the blockchain network, hash information corresponding to the stored medical data.

5. The method of claim 1, wherein the de-identifying of the read medical data and providing the de-identified medical data to the other user comprises:
converting the read medical data into digital rights management (DRM) medical data;
converting the DRM medical data into a preset common data format (CDF);
de-identifying personal information included in the DRM medical data converted into the CDF; and
encrypting the DRM medical data and providing the encrypted DRM medical data to the other user.

6. The method of claim 1, further comprising:
registering a development model developed based on medical data from the other user; and
setting the registered development model and a previously registered development model so that the registered development model and the previously registered development model are able to be distributed.

7. The method of claim 6, further comprising providing incentives managed in the blockchain network to the other user who has registered the development model when the registered development model from the other user is distributed based on a purchase behavior of another third user.

8. The method of claim 1, further comprising:
receiving an inquiry request for medical data provided by the user among the stored medical data;
reading, from the blockchain network, the medical data corresponding to the inquiry request; and
providing the read medical data to the user.

9. A server for providing a user-sovereign medical data sharing and transaction service, the server comprising:
a medical data collection unit configured to collect medical data generated from at least one of a user and a medical institution;
a blockchain unit configured to store the collected medical data in a blockchain network while operating in conjunction with the blockchain network; and
an artificial intelligence unit configured to read, from the blockchain network, medical data corresponding to the use request when receiving a use request for the stored medical data from another user, de-identify the read medical data, and provide the de-identified medical data to the other user.

10. The server of claim 9, wherein:
the medical data collection unit receives sensing data of a wearable device of the user or examination data in a medical institution that the user has visited through an application previously installed in a terminal of the user, and
the artificial intelligence unit provides the user with incentives managed in the blockchain network when receiving the medical data.

11. The server of claim 9, wherein:
the blockchain unit stores, in an external distributed repository, medical data satisfying a preset condition, and stores, in the blockchain network, hash information corresponding to the stored medical data, and
the preset condition comprises at least one of a type and amount of the medical data.

12. The server of claim 9, further comprising:
a use right management unit configured to convert the read medical data into digital rights management (DRM) medical data; and
a data processing unit configured to convert the DRM medical data into a preset common data format (CDF), de-identify personal information included in the DRM medical data converted into the CDF, and encrypt the DRM medical data,
wherein the artificial intelligence unit provides the encrypted DRM medical data to the other user.

13. The server of claim 9, wherein the artificial intelligence unit registers a development model developed based on medical data from the other user, sets the registered development model and a previously registered development model so that the registered development model and the previously registered development model are able to be distributed, and provides incentives managed in the blockchain network to the other user who has registered the development model when the registered development model from the other user is distributed based on a purchase behavior of another third user.

14. The server of claim 9, wherein when receiving an inquiry request for medical data provided by the user among the stored medical data, the artificial intelligence unit reads, from the blockchain network, the medical data corresponding to the inquiry request, and provides the read medical data to the user.

15. A server for providing a user-sovereign medical data sharing and transaction service, the server comprising:
a communication module configured to transmit and receive data to and from a terminal of a user and a terminal of a medical institution;
a memory in which a program for providing a medical data sharing and transaction service based on medical data generated from at least one of the user and the medical institution has been stored; and
a processor configured to collect and store the medical data when the program stored in the memory is executed and to read medical data corresponding to a use request when receiving the use request for the stored medical data from another user, de-identify the read medical data, and provide the de-identified medical data to the other user.
